(19) United States
(12) Patent Application Publication
(10) Pub. No.: US 2026/0009778 A1
(43) Pub. Date: Jan. 8, 2026

(54) MULTI-SEQUENCE SEAWATER SAMPLING APPARATUS AND METHOD WITH THERMAL INSULATION AND PRESSURE RETENTION

(71) Applicants: GUANGDONG LABORATORY OF SOUTHERN OCEAN SCIENCE AND ENGINEERING (GUANGZHOU), Guangdong (CN); GUANGDONG UNIVERSITY OF TECHNOLOGY, Guangdong (CN)

(72) Inventors: Jingchun FENG, Guangdong (CN); Si ZHANG, Guangdong (CN); Yan XIE, Guangdong (CN); Zhifeng YANG, Guangdong (CN)

(73) Assignees: GUANGDONG LABORATORY OF SOUTHERN OCEAN SCIENCE AND ENGINEERING (GUANGZHOU), Guangdong (CN); GUANGDONG UNIVERSITY OF TECHNOLOGY, Guangdong (CN)

(21) Appl. No.: 19/260,304

(22) Filed: Jul. 4, 2025

(30) Foreign Application Priority Data
Jul. 5, 2024 (CN) 202410896781.0

(51) Int. Cl. G01N 33/18; G01N 1/10; G01N 1/14; G01N 1/16

(52) U.S. Cl. CPC G01N 33/1886; G01N 1/14; G01N 1/16; G01N 2001/1062

(57) ABSTRACT

The present invention discloses a multi-sequence seawater sampling apparatus and method with thermal insulation and pressure retention, relating to the technical field of marine microorganism sampling. The apparatus includes an outer frame, a flow velocity regulation unit, a rotation unit, a multi-sequence sampling unit, and a control unit. The multi-sequence sampling unit is disposed in the outer frame and includes a plurality of sampling modules. Each of the sampling modules includes a sampling valve, a sampling bottle, a gas phase shutoff valve, and a back pressure valve that are connected in sequence. The plurality of sampling valves are circumferentially distributed at a top of the outer frame, and control ends of all the sampling valves face the rotation unit. The rotation unit is disposed at a center of the top of the outer frame, and an end of the rotation unit abuts against the control end of the sampling valve.

### CROSS REFERENCE TO THE RELATED APPLICATIONS

This application claims the priority benefit of China application serial no. 202410896781.0, filed on Jul. 5, 2024. The entirety of the above-mentioned patent applications is hereby incorporated by reference herein and made a part of this specification.

### BACKGROUND

### Technical Field

The present invention relates to the technical field of marine microorganism sampling, and more specifically, to a multi-sequence seawater sampling apparatus and method with thermal insulation and pressure retention.

### Description of Related Art

90% of marine organisms are marine microorganisms, which participate in the cycling of key elements such as carbon, nitrogen, and sulfur, forming an indispensable component for maintaining the normal functioning of marine ecosystems. The widespread deep-sea methane seepage phenomenon in the seafloor results in a significant methane source, providing an important carbon source for marine microorganisms. In regions of methane seepage in the seafloor, due to variations in vertical distance from the seepage source and the influence of different temperature and pressure conditions at various ocean depths, the dissolved methane content varies across different ocean layers. Additionally, under the methane metabolism of microorganisms, significant changes occur in the content of various other elements in seawater. Exploring the mechanisms by which different ocean layers affect microbial methane metabolism is of great significance for revealing the marine carbon cycle.

Due to the challenges of accessing the deep sea and conducting long-cycle in-situ marine studies, sampling microorganisms from seawater at different ocean layers and studying their methane metabolism processes are important approaches to elucidate methane metabolism mechanisms, which rely on corresponding multi-layer seawater sampling equipment. However, current equipment for multi-layer seawater sampling primarily consists of conventional Conductivity, Temperature, Depth (CTD) measurement systems, which are incapable of thermal insulation and pressure retention. Due to temperature and pressure changes, dissolved gases in seawater may escape, and microorganisms may experience varying degrees of deactivation due to environmental changes, causing the sampled microorganisms to deviate from their in-situ environment, thus making it impossible to reconstruct the true methane metabolism process. To address these issues, numerous single-sequence seawater sampling devices with thermal insulation, pressure retention, or both have been developed, but due to their single-sequence nature, they cannot meet the needs for multi-depth sequence sampling. Although multiple single-sequence samplings can achieve a multi-sequence effect, repeated dives result in large sampling errors and low sampling efficiency. The limited existing studies on multi-sequence seawater sampling with thermal insulation and pressure retention can only achieve passive thermal insulation of seawater, with poor accuracy in thermal insulation capabilities. Additionally, during the pressure-retaining seawater sampling process, passive sampling of seawater from different layers is achieved primarily through the pressure difference between the seawater and the sampling bottle. However, the large pressure difference between the seawater environment and the sampling bottle and the high flow rate lead to brief escape of dissolved gases and phased distortion of microorganisms.

### SUMMARY

To overcome the defects in the existing seawater sampling process, where passive sampling based on high pressure differences leads to phased distortion of dissolved gases and microbial properties in seawater, the present invention provides a multi-sequence seawater sampling apparatus and method with thermal insulation and pressure retention. It enables slow, isobaric injection of seawater from multiple target water layers, ensuring sampling stability and efficiency, reducing sampling errors, and providing significant support for exploring the depth-dependent characteristics of marine microorganisms.

To address the above technical problems, the technical solution of the present invention is as follows:

The present invention provides a multi-sequence seawater sampling apparatus with thermal insulation and pressure retention, including an outer frame, a flow velocity regulation unit, a rotation unit, a multi-sequence sampling unit, and a control unit.

The multi-sequence sampling unit is disposed in the outer frame and includes a plurality of sampling modules. Each of the sampling modules includes a sampling valve, a sampling bottle, a gas phase shutoff valve, and a back pressure valve that are connected in sequence. The plurality of sampling valves are circumferentially distributed at a top of the outer frame, and control ends of the sampling valves all face the rotation unit.

The rotation unit is disposed at a center of the top of the outer frame, and an end of the rotation unit abuts against the control end of the sampling valve.

The flow velocity regulation unit is disposed in the outer frame and includes a sampling injection pump, an automatic shutoff valve, a flow rate controller, and a first multi-channel distribution valve that are connected in sequence, where each water outlet end of the first multi-channel distribution valve is connected to a water inlet end of the sampling valve of one sampling module correspondingly.

The control unit is disposed in the outer frame, and the output end of the control unit is connected to control ends of the rotation unit, the sampling bottle, the sampling injection pump, and the flow rate controller.

In the sampling apparatus provided by the present invention, the outer frame is configured to support other units. The rotation unit is disposed at the center of the top of the outer frame, with the plurality of sampling valves circumferentially distributed at the top of the outer frame, control ends of all the sampling valves facing the rotation unit, and an end of the rotation unit abutting against the control end of the sampling valve. The rotation unit can set different rotation angles based on the number of sampling valves. When any one of sampling valves needs to be opened, the rotation unit is rotated to a preset angle to open the sampling valve through mechanical compression. Before the sampling apparatus is lowered into the water, water depths and corresponding environmental pressures of a plurality of target water sampling layers are determined. According to the sequence of the target water sampling layers from deep to shallow, nitrogen gas is pre-charged into gas phase chambers of the plurality of sampling bottles via the gas phase shutoff valves to pressure values equal to the environmental pressures, and corresponding back pressure valves are adjusted to pressure values equal to the environmental pressures, keeping the gas phase shutoff valves in an open state. Charging the pressure of the gas phase chamber of the sampling bottle to be equal to the pressure of the target water sampling layer reduces the pressure difference between the external seawater environment and the sampling bottle, avoiding rapid seawater inflow into the liquid phase chamber of the sampling bottle due to excessive pressure differences. The back pressure valve is used during seawater injection to maintain the system pressure inside the sampling bottle constant and equal to the pressure of the target water sampling layer. The sampling apparatus is lowered to the target water sampling layer, the sampling injection pump and the automatic shutoff valve are controlled to be activated, and a parameter of the flow rate controller is set to control the injected seawater volume, allowing seawater to be slowly and isobarically injected into the liquid phase chamber of the sampling bottle. Since the pressure of the gas phase chamber of the sampling bottle is equal to the external hydrostatic pressure, and the back pressure of the back pressure valve is also equal to the pressure of the gas phase chamber, the sampling injection pump only needs to provide minimal additional injection force. When the seawater volume reaches the preset target value, the automatic shutoff valve closes automatically to prevent further seawater injection. After the automatic shutoff valve closes, the sampling injection pump stops working due to current self-protection caused by the blocked inlet, and the flow rate controller also stops with the closure of the automatic shutoff valve. The rotation unit rotates away from the position of the current sampling valve, and the current sampling valve closes. This process is repeated to sample multiple target water sampling layers.

Preferably, the rotation unit includes a rotation actuator and a cam; and the rotation actuator is disposed at the center of the top of the outer frame, the cam is disposed on the rotation actuator, and an end of the cam abuts against the control end of the sampling valve.

Preferably, each of the sampling bottles includes an upper end cap, an outer bottle wall, an inner bottle wall, a piston, a plurality of cooling heat-exchange modules, a circulation pipeline, a seawater circulation inlet, a seawater circulation outlet, and a lower end cap.

The upper end cap is disposed at one end of the outer bottle wall, the lower end cap is disposed at the other end of the outer bottle wall, the inner bottle wall is concentrically disposed with the outer bottle wall, and a vacuum thermal insulation layer is formed between the outer bottle wall and the inner bottle wall. The piston is disposed in the inner bottle wall and divides a cavity between the inner bottle wall, the upper end cap, and the lower end cap into a liquid phase chamber and a gas phase chamber.

The outer bottle wall is provided with the seawater circulation inlet and the seawater circulation outlet that are communicated with each other, and the plurality of cooling heat-exchange modules are uniformly distributed on an outer wall surface of the inner bottle wall. The seawater circulation inlet, the plurality of cooling heat-exchange modules, and the seawater circulation outlet form series connection via the circulation pipeline.

A water outlet end of the sampling valve is connected to the upper end cap of the sampling bottle, and the lower end cap of the sampling bottle is connected to the gas phase shutoff valve.

The control ends of the plurality of cooling heat-exchange modules are all connected to the output end of the control unit.

Each of the sampling bottles simultaneously performs active thermal insulation and passive thermal insulation. Passive thermal insulation is achieved via the vacuum thermal insulation layer between the outer bottle wall and the inner bottle wall, increasing thermal resistance. In addition, in the vacuum thermal insulation layer, the plurality of cooling heat-exchange modules are uniformly distributed on the outer wall surface of the inner bottle wall for active thermal insulation. Before the sampling apparatus is lowered into the water, temperatures of the plurality of target water sampling layers are determined and correspondingly set as target temperatures of the cooling heat-exchange modules of the plurality of sampling bottles. The cooling heat-exchange modules in different sampling bottles automatically control the temperature based on the target temperatures. The combination of active cooling and vacuum thermal insulation achieves efficient thermal insulation of seawater.

Preferably, each of the cooling heat-exchange modules includes a semiconductor cooling chip, a semiconductor heat exchange chip, and a semiconductor heat-exchange water tank.

A cooling end of the semiconductor cooling chip is disposed on the outer wall surface of the inner bottle wall, a heat dissipation end of the semiconductor cooling chip is connected to one end of the semiconductor heat exchange chip, and the other end of the semiconductor heat exchange chip is connected to the semiconductor heat-exchange water tank.

The semiconductor heat-exchange water tank is provided with a first port and a second port.

A series connection path is formed by arranging the circulation pipeline between the first port of the semiconductor heat-exchange water tank of one cooling heat-exchange module and the second port of the semiconductor heat-exchange water tank of another cooling heat-exchange module, the first port of the semiconductor heat-exchange water tank of a cooling heat-exchange module at one end of the series connection path is connected to the seawater circulation inlet via the circulation pipeline, and the second port of the semiconductor heat-exchange water tank of a cooling heat-exchange module at the other end of the series connection path is connected to the seawater circulation outlet via the circulation pipeline.

The control end of the semiconductor cooling chip is connected to the output end of the control unit.

Preferably, the apparatus further includes a seawater circulation heat exchange unit, and the seawater circulation heat exchange unit includes circulation injection pump, a second multi-channel distribution valve, a third multi-channel distribution valve, a plurality of seawater inlet pipes, and a plurality of seawater outlet pipes.

Both a first water inlet and a first water outlet of the circulation injection pump are suspended.

The second water outlet of the circulation injection pump is connected to a water inlet end of the second multi-channel distribution valve, each water outlet end of the second multi-channel distribution valve is connected to one end of one seawater inlet pipe, and the other end of each of the seawater inlet pipes is connected to the seawater circulation inlet of one sampling bottle correspondingly.

The second water inlet of the circulation injection pump is connected to a water outlet end of the third multi-channel distribution valve, each water inlet end of the third multi-channel distribution valve is connected to one end of one seawater outlet pipe, and the other end of each of the seawater outlet pipes is connected to the seawater circulation outlet of one sampling bottle correspondingly.

The output end of the control unit is connected to the control end of the circulation injection pump.

The cooling end of the semiconductor cooling chip is closely attached to the outer wall surface of the inner bottle wall. The heat dissipation end of the semiconductor cooling chip conducts heat through a semiconductor heat exchange chip, and the semiconductor heat exchange chip transfers heat with external circulating seawater using a semiconductor heat-exchange water tank. The seawater circulation heat exchange unit enables a single circulation injection pump to provide circulating seawater for the cooling heat-exchange modules of the multiple sampling bottles. The cooling heat-exchange modules of different sampling bottles are connected in parallel via the second multi-channel distribution valve and the third multi-channel distribution valve.

During the circulating water flow process, seawater first enters the circulation injection pump via the first water inlet of the circulation injection pump and then flows out via the second water outlet of the circulation injection pump. The outflowing seawater forms multiple channeled seawater flows in the second multi-channel distribution valve and then enters the cooling heat-exchange modules respectively via the seawater inlet pipes are connected to the seawater circulation inlets of different sampling bottles. For multiple cooling heat-exchange modules on a single sampling bottle, the circulating seawater is reused for heat exchange through series connection via the circulation pipeline. The seawater after heat exchange flows out via the seawater circulation outlet of the sampling bottle. The seawater flowing out from different sampling bottles is collected in the third multi-channel distribution valve via the seawater outlet pipes, flows into the circulation injection pump via the second water inlet, and is finally discharged via the first water outlet. The seawater circulation heat exchange unit and cooling heat-exchange modules are activated immediately after the apparatus is lowered into the water, enabling different sampling bottles to quickly reach and stabilize at the temperature of the target seawater layer.

Preferably, each of the sampling modules further includes a liquid phase shutoff valve.

A water outlet end of the sampling valve is connected to one end of the liquid phase shutoff valve, and the other end of the liquid phase shutoff valve is connected to the sampling bottle.

The liquid phase shutoff valve is configured to cut off the connection between the liquid phase chamber of the sampling bottle and the front end of the liquid phase shutoff valve after the sampling apparatus is retrieved to the ship, reducing the probability of leakage. At the same time, in addition to its role in pre-charging nitrogen, the gas phase shutoff valve is configured to cut off the connection between the gas phase chamber of the sampling bottle and pipelines and water at the rear end of the sampling bottle, reducing the probability of leakage.

Preferably, each of the sampling modules further includes a first check valve and a second check valve.

The water outlet end of the sampling valve is connected to one end of the first check valve, and the other end of the first check valve is connected to one end of the liquid phase shutoff valve.

The other end of the back pressure valve is connected to one end of the second check valve, and the other end of the second check valve is suspended.

The first check valve is configured to further prevent leakage in the sampling bottle caused by leakage at its front end, and the second check valve is configured to prevent seawater from backflowing into the gas phase chamber of the sampling bottle from the bottom.

Preferably, each of the sampling modules further includes a temperature sensor, a liquid phase pressure sensor, and a gas phase pressure sensor.

The temperature sensor and the liquid phase pressure sensor are both disposed at the upper end cap, the gas phase pressure sensor is disposed at the lower end cap.

Data output ends of the temperature sensor, the liquid phase pressure sensor, and the gas phase pressure sensor are all connected to a data input end of the control unit.

The temperature sensor is configured to monitor the temperature in the liquid phase chamber of the sampling bottle, providing feedback for the temperature control of the semiconductor cooling chip. The liquid phase pressure sensor is configured to monitor the pressure in the liquid phase chamber of the sampling bottle, determining whether there is a leakage in the sampling bottle, and also providing feedback for the injection of the target pressure in the gas phase chamber and the seawater injection by the flow rate controller. The gas phase pressure sensor is configured to monitor the pressure in the gas phase chamber of the sampling bottle.

The present invention further provides a multi-sequence seawater sampling method with thermal insulation and pressure retention, applied to the above-mentioned sampling apparatus, and including:
S1: determining water depths, and corresponding environmental pressures and temperatures of a plurality of target water sampling layers, pre-charging nitrogen gas into the gas phase chambers of the plurality of sampling bottles correspondingly via the gas phase shutoff valves to pressure values equal to the environmental pressures based on a sequence of the water depths of the target water sampling layers, adjusting corresponding back pressure valves to pressure values equal to the environmental pressures, and maintaining the gas phase shutoff valves in an open state;
S2: setting target temperatures of all the semiconductor cooling chips in corresponding sampling bottles based on the temperatures of the target water sampling layers;
S3: during submersion of the sampling apparatus into water, controlling, by the control unit, the semiconductor cooling chips and the circulation injection pump to be activated, and lowering the sampling apparatus to a target water sampling layer with a deepest water depth;
S4: controlling the rotation actuator to drive the cam to rotate to the control end of a corresponding sampling valve, and opening the sampling valve through mechanical compression;
S5: setting a parameter of the flow rate controller correspondingly, controlling the sampling injection pump and the automatic shutoff valve to be activated, injecting seawater into the opened sampling valve via the first multi-channel distribution valve, further introducing the seawater into the liquid phase chamber of a corresponding sampling bottle, and acquiring, by the temperature sensor, the liquid phase pressure sensor, and the gas phase pressure sensor, a current temperature, a current liquid phase pressure, and a current gas phase pressure of the sampling bottle in real time;
S6: measuring, by the flow rate controller, an injected seawater volume in real time, controlling the automatic shutoff valve, the sampling injection pump, and the flow rate controller to be deactivated sequentially when the seawater volume reaches a preset target value, and controlling the rotation actuator to drive the cam to rotate to move away from the control end of the current sampling valve; and
S7: determining whether water sampling for all the target water sampling layers is completed; and if the water sampling is not completed, lifting the sampling apparatus to a next target water sampling layer and repeating steps S4 to S6; otherwise, terminating the water sampling.

Preferably, after the lowering the sampling apparatus to a target water sampling layer with a deepest water depth, the method further includes:
introducing the seawater via the first water inlet of the circulation injection pump and discharging the seawater via the second water outlet; distributing the discharged seawater into a plurality of channeled seawater flows via the second multi-channel distribution valve, and introducing the plurality of channeled seawater flows into the cooling heat-exchange modules of different sampling bottles through the seawater inlet pipes from the seawater circulation inlets; and
collecting the seawater, after heat exchange through all the cooling heat-exchange modules of the sampling bottles, from the seawater circulation outlets into the third multi-channel distribution valve via the seawater outlet pipes; and introducing the collected seawater into the circulation injection pump via the second water inlet and discharging the seawater via the first water outlet.

As compared with the prior art, the technical solution of the present invention has the following beneficial effects:

In the present invention, the rotation unit is disposed at the center of the top of the outer frame, with the plurality of sampling valves circumferentially distributed at the top of the outer frame, the control ends of all the sampling valves facing the rotation unit, and an end of the rotation unit abutting against the control end of the sampling valve. Before the sampling apparatus is lowered into the water, the water depths and corresponding environmental pressures of the plurality of target water sampling layers are determined. The nitrogen gas is pre-charged into the gas phase chambers of the plurality of sampling bottles via the gas phase shutoff valves to pressure values equal to the environmental pressures, and the corresponding back pressure valves are adjusted to pressure values equal to the environmental pressures, keeping the gas phase shutoff valves in the open state. Charging the pressure of the gas phase chamber of the sampling bottle to be equal to the pressure of the target water sampling layer reduces the pressure difference between the external seawater environment and the sampling bottle, avoiding rapid seawater inflow into the liquid phase chamber of the sampling bottle due to excessive pressure differences. The back pressure valve is used during seawater injection to maintain the system pressure inside the sampling bottle constant and equal to the pressure of the target water sampling layer. The sampling apparatus is lowered to the target water sampling layer, the rotation unit is rotated to the control end of the corresponding sampling valve, the sampling valve is opened through mechanical compression, the sampling injection pump and the automatic shutoff valve are controlled to be activated, and the parameter of the flow rate controller is set to control the injected seawater volume, allowing the seawater to be slowly and isobarically injected into the liquid phase chamber of the sampling bottle. The automatic shutoff valve, the sampling injection pump, and the flow rate controller are controlled be to deactivated sequentially when the seawater volume reaches the preset target value, and the rotation actuator is controlled to drive the cam to rotate to move away from the control end of the current sampling valve. The present invention enables slow, isobaric injection of seawater from multiple target water layers, ensuring sampling stability and efficiency, reducing sampling errors, and providing significant support for exploring the depth-dependent characteristics of marine microorganisms.

### BRIEF DESCRIPTION OF THE DRAWINGS

FIG. 1 is a schematic structural diagram of a multi-sequence seawater sampling apparatus with thermal insulation and pressure retention according to Embodiment 1.

FIG. 2 is a schematic structural diagram of a multi-sequence seawater sampling apparatus with thermal insulation and pressure retention according to Embodiment 2.

FIG. 3 is a cross-sectional view of a sampling bottle according to Embodiment 2.

FIG. 4 is a top view of a rotation unit according to Embodiment 2.

FIG. 5 is a schematic structural diagram of a seawater circulation heat exchange unit according to Embodiment 2.

FIG. 6A and FIG. 6B are a flowchart of a multi-sequence seawater sampling method with thermal insulation and pressure retention according to Embodiment 3.

### DESCRIPTION OF THE EMBODIMENTS

The drawings are for illustrative purposes only and should not be construed as limiting this patent.

To better illustrate embodiments, certain components in the drawings may be omitted, enlarged, or reduced, and do not represent the actual product dimensions.

For those skilled in the art, it is understandable that certain well-known structures in the drawings and their descriptions may be omitted.

The technical solution of the present invention is further described below with reference to the drawings and embodiments.

### Embodiment 1

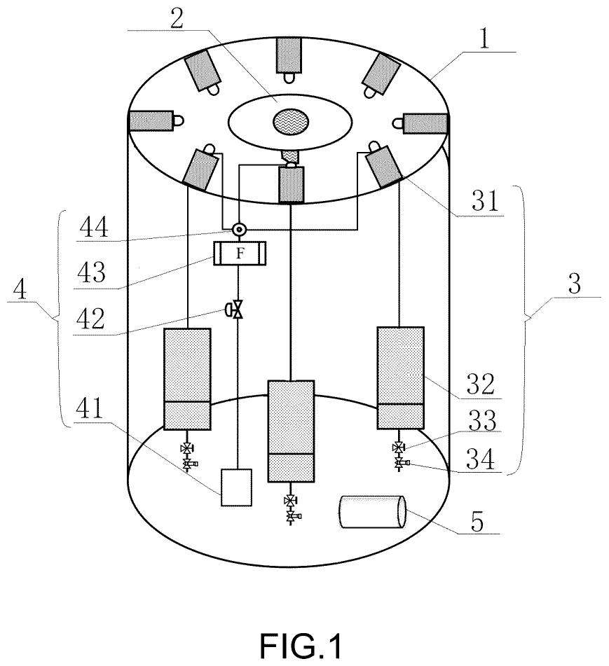

This embodiment provides a multi-sequence seawater sampling apparatus with thermal insulation and pressure retention, as shown in FIG. 1, including an outer frame 1, a rotation unit 2, a multi-sequence sampling unit, a flow velocity regulation unit 4, and a control unit 5.

The multi-sequence sampling unit is disposed in the outer frame 1 and includes a plurality of sampling modules 3. Each of the sampling modules 3 includes a sampling valve 31, a sampling bottle 32, a gas phase shutoff valve 33, and a back pressure valve 34 that are connected in sequence. The plurality of sampling valves 31 are circumferentially distributed at a top of the outer frame 1, and control ends of all the sampling valves 31 face the rotation unit 2.

The rotation unit 2 is disposed at a center of the top of the outer frame 1, and an end of the rotation unit 2 abuts against the control end of the sampling valve 31.

The flow velocity regulation unit 4 is disposed in the outer frame 1 and includes a sampling injection pump 41, an automatic shutoff valve 42, a flow rate controller 43, and a first multi-channel distribution valve 44 that are connected in sequence, where each water outlet end of the first multi-channel distribution valve 44 is connected to a water inlet end of the sampling valve 31 of one sampling module 3 correspondingly.

The control unit 5 is disposed in the outer frame 1, and the output end of the control unit 5 is connected to control ends of the rotation unit 2, the sampling bottle 32, the sampling injection pump 41, and the flow rate controller 43.

In a specific implementation process, the outer frame 1 is configured to support other units. The rotation unit 2 is disposed at the center of the top of the outer frame 1, with the plurality of sampling valves 31 circumferentially distributed at the top of the outer frame 1. Control ends of the all sampling valves 31 face the rotation unit 2, and an end of the rotation unit 2 abuts against the control end of the sampling valve 31. The rotation unit 2 can set different rotation angles based on the number of sampling valves 31. When any one of sampling valves 31 needs to be opened, the rotation unit 2 is rotated to a preset angle to open the sampling valve 31 through mechanical compression. Before the sampling apparatus is lowered into the water, the water depths and corresponding environmental pressures of the plurality of target water sampling layers are determined. According to the sequence of the target water sampling layers from deep to shallow, nitrogen gas is pre-charged into gas phase chambers of the plurality of sampling bottles 32 via the gas phase shutoff valves 33 until pressure values equal to the environmental pressures, and corresponding back pressure valves 34 are adjusted to pressure values equal to the environmental pressures, keeping the gas phase shutoff valves 33 in an open state. Charging the pressure of the gas phase chamber of the sampling bottle 32 to be equal to the pressure of the target water sampling layer reduces the pressure difference between the external seawater environment and the sampling bottle 32, avoiding rapid seawater inflow into the liquid phase chamber of the sampling bottle 32 due to excessive pressure differences. The back pressure valve 34 is used during seawater injection to maintain the system pressure inside the sampling bottle 32 constant and equal to the pressure of the target water sampling layer. The sampling apparatus is lowered to the target water sampling layer, the sampling injection pump 41 and the automatic shutoff valve 42 are controlled to be activated, and a parameter of the flow rate controller 43 is set to control the injected seawater volume, allowing seawater to be slowly and isobarically injected into the liquid phase chamber of the sampling bottle 32. Since the pressure of the gas phase chamber of the sampling bottle 32 is equal to the external hydrostatic pressure, and the back pressure of the back pressure valve 34 is also equal to the pressure of the gas phase chamber, the sampling injection pump 41 only needs to provide minimal additional injection force. When the seawater volume reaches the preset target value, the automatic shutoff valve 42 closes automatically to prevent further seawater injection. After the automatic shutoff valve 42 closes, the sampling injection pump 41 stops working due to current self-protection caused by the blocked inlet, and the flow rate controller 43 also stops with the closure of the automatic shutoff valve 42. The rotation unit 2 rotates away from the position of the current sampling valve 31, and the current sampling valve 31 closes. This process is repeated to sample multiple target water sampling layers.

### Embodiment 2

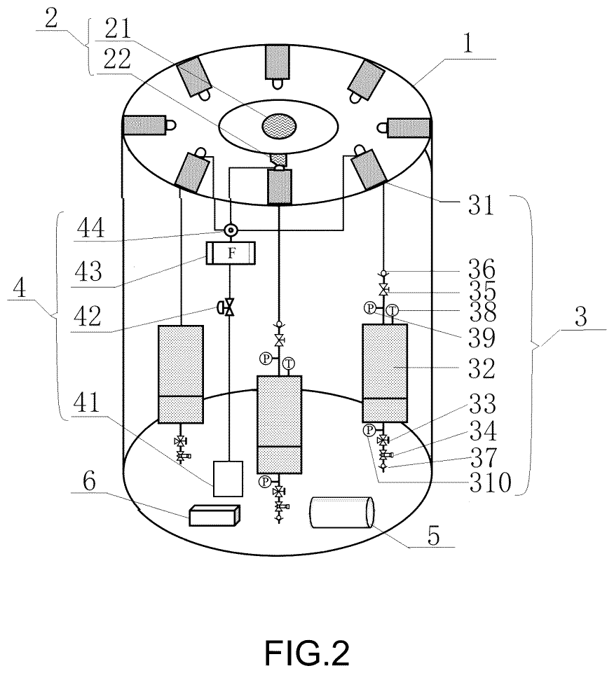

This embodiment provides a multi-sequence seawater sampling apparatus with thermal insulation and pressure retention, as shown in FIG. 2, including an outer frame 1, a flow velocity regulation unit 4, a rotation unit 2, a multi-sequence sampling unit, a control unit 5, and a seawater circulation heat exchange unit 6.

The multi-sequence sampling unit is disposed in the outer frame 1 and includes a plurality of sampling modules 3. Each of the sampling modules 3 includes a sampling valve 31, a first check valve 36, a liquid phase shutoff valve 35, a sampling bottle 32, a gas phase shutoff valve 33, a back pressure valve 34, and a second check valve 37 that are connected in sequence. The plurality of sampling valves 31 are circumferentially distributed at a top of the outer frame 1, and control ends of all the sampling valves 31 face the rotation unit 2.

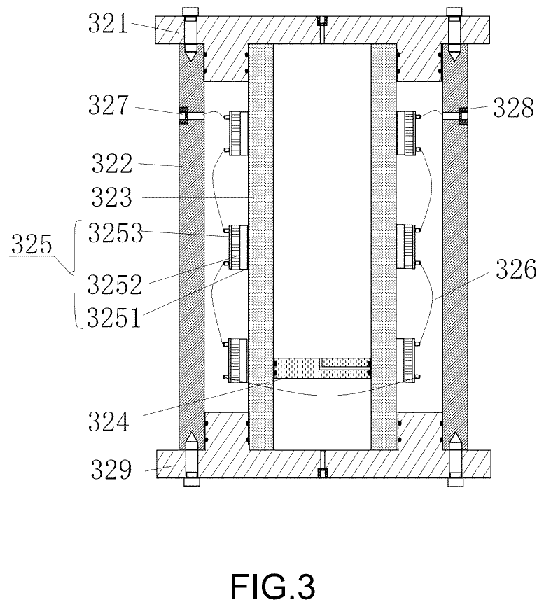

As shown in FIG. 3, each of the sampling bottles 32 includes an upper end cap 321, an outer bottle wall 322, an inner bottle wall 323, a piston 324, a plurality of cooling heat-exchange modules 325, a circulation pipeline 326, a seawater circulation inlet 327, a seawater circulation outlet 328, and a lower end cap 329.

The upper end cap 321 is disposed at one end of the outer bottle wall 322, the lower end cap 329 is disposed at the other end of the outer bottle wall 322, the inner bottle wall 323 is concentrically disposed with the outer bottle wall 322, and a vacuum thermal insulation layer is formed between the outer bottle wall 322 and the inner bottle wall 323. The piston 324 is disposed in the inner bottle wall 323 and divides a cavity between the inner bottle wall 323, the upper end cap 321, and the lower end cap 329 into a liquid phase chamber and a gas phase chamber.

The outer bottle wall 322 is provided with the seawater circulation inlet 327 and the seawater circulation outlet 328 that are communicated with each other, and the plurality of cooling heat-exchange modules 325 are uniformly distributed on an outer wall surface of the inner bottle wall 323. The seawater circulation inlet 327, the plurality of cooling heat-exchange modules 325, and the seawater circulation outlet 328 form series connection via the circulation pipeline 326.

Each of the cooling heat-exchange modules 325 further includes a semiconductor cooling chip 3251, a semiconductor heat exchange chip 3252, and a semiconductor heat-exchange water tank 3253.

A cooling end of the semiconductor cooling chip 3251 is disposed on the outer wall surface of the inner bottle wall 323, a heat dissipation end of the semiconductor cooling chip 3251 is connected to one end of the semiconductor heat exchange chip 3252, and the other end of the semiconductor heat exchange chip 3252 is connected to the semiconductor heat-exchange water tank 3253.

The semiconductor heat-exchange water tank 3253 is provided with a first port and a second port.

A series connection path is formed by arranging the circulation pipeline 326 between the first port of the semiconductor heat-exchange water tank 3253 of a cooling heat-exchange module 325 and the second port of the semiconductor heat-exchange water tank 3253 of another cooling heat-exchange module 325. The first port of the semiconductor heat-exchange water tank 3253 of a cooling heat-exchange module 325 at one end of the series connection path is connected to the seawater circulation inlet 327 via the circulation pipeline 326, and the second port of the semiconductor heat-exchange water tank 3253 of a cooling heat-exchange module 325 at the other end of the series connection path is connected to the seawater circulation outlet 328 via the circulation pipeline 326.

The control end of the semiconductor cooling chip 3251 is connected to the output end of the control unit 5.

Each of the sampling modules 3 further includes a temperature sensor 38, a liquid phase pressure sensor 39, and a gas phase pressure sensor 310. The temperature sensor 38 and the liquid phase pressure sensor 39 are both disposed at the upper end cap 321, and the gas phase pressure sensor 310 is disposed at the lower end cap 329.

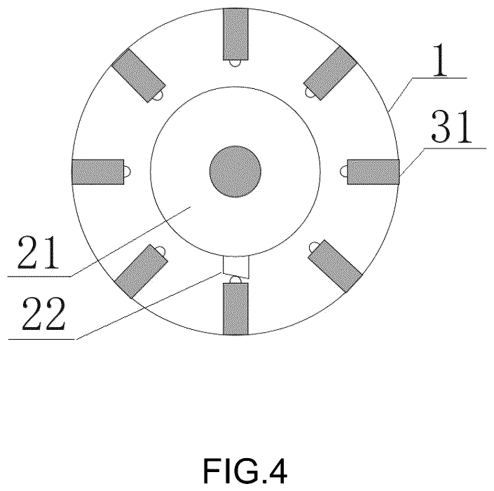

As shown in FIG. 4, the rotation unit 2 includes a rotation actuator 21 and a cam 22; the rotation actuator 21 is disposed at the center of the top of the outer frame 1, the cam 22 is disposed on the rotation actuator 21, and an end of the cam 22 abuts against the control end of the sampling valve 31.

The flow velocity regulation unit 4 is disposed in the outer frame 1 and includes a sampling injection pump 41, an automatic shutoff valve 42, a flow rate controller 43, and a first multi-channel distribution valve 44 that are connected in sequence, where each water outlet end of the first multi-channel distribution valve 44 is connected to a water inlet end of the sampling valve 31 of one sampling module 3 correspondingly.

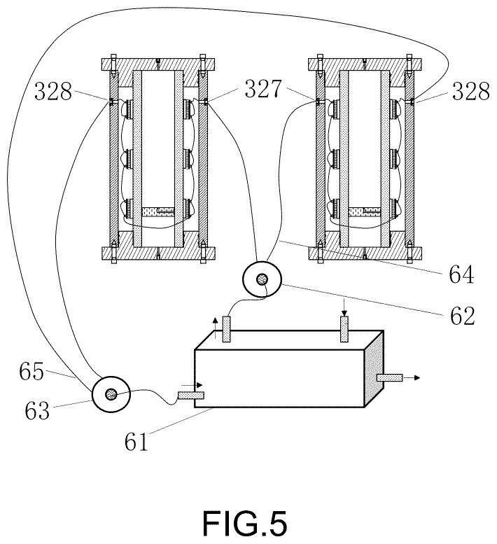

As shown in FIG. 5, the seawater circulation heat exchange unit 6 includes a circulation injection pump 61, a second multi-channel distribution valve 62, a third multi-channel distribution valve 63, a plurality of seawater inlet pipes 64, and a plurality of seawater outlet pipes 65.

A first water inlet and a first water outlet of the circulation injection pump 61 are suspended.

The second water outlet of the circulation injection pump 61 is connected to a water inlet end of the second multi-channel distribution valve 62, each water outlet end of the second multi-channel distribution valve 62 is connected to one end of one seawater inlet pipe 64, and the other end of each of the seawater inlet pipes 64 is connected to the seawater circulation inlet 327 of one sampling bottle 32 correspondingly.

The second water inlet of the circulation injection pump 61 is connected to a water outlet end of the third multi-channel distribution valve 63, each water inlet end of the third multi-channel distribution valve 63 is connected to one end of one seawater outlet pipe 65, and the other end of each of the seawater outlet pipes 65 is connected to the seawater circulation outlet 328 of one sampling bottle 32 correspondingly.

The control unit 5 is disposed in the outer frame 1, and the output end of the control unit 5 is connected to the control ends of the rotation actuator 21, the sampling injection pump 41, the flow rate controller 43, and the circulation injection pump 61. The data output ends of the temperature sensor 38, the liquid phase pressure sensor 39, and the gas phase pressure sensor 310 are all connected to the data input end of the control unit 5.

In a specific implementation process, the outer frame 1 is configured to support other units. The rotation actuator 21 is disposed at the center of the top of the outer frame 1, with a plurality of sampling valves 31 circumferentially distributed at the top of the outer frame 1, the control ends of all the sampling valves 31 facing the rotation actuator 21, and an end of the cam 22 abutting against the control end of the sampling valve 31. The rotation actuator 21 can set different rotation angles based on the number of sampling valves 31. When any one of sampling valve 31 needs to be opened, the rotation actuator 21 is rotated to a preset angle to open the sampling valve 31 through mechanical compression. Before the sampling apparatus is lowered into the water, water depths and corresponding environmental pressures of a plurality of target water sampling layers are determined. According to the sequence of the target water sampling layers from deep to shallow, nitrogen gas is pre-charged into the gas phase chambers of the plurality of sampling bottles 32 via the gas phase shutoff valves 33 to pressure values equal to the environmental pressures, and the corresponding back pressure valves 34 are adjusted to pressure values equal to the environmental pressures, keeping the gas phase shutoff valves 33 in an open state. Charging the pressure of the gas phase chamber of the sampling bottle 32 to be equal to the pressure of the target water sampling layer reduces the pressure difference between the external seawater environment and the sampling bottle 32, avoiding rapid seawater inflow into the liquid phase chamber of the sampling bottle 32 due to excessive pressure differences. The back pressure valve 34 is used during seawater injection to maintain the system pressure inside the sampling bottle 32 constant and equal to the pressure of the target water sampling layer.

Each of the sampling bottles 32 simultaneously performs active and passive thermal insulation. Passive thermal insulation is achieved through a vacuum thermal insulation layer between the outer bottle wall 322 and the inner bottle wall 323, increasing thermal resistance. In addition, the plurality of cooling heat-exchange modules 325 are uniformly distributed on the outer wall surface of the inner bottle wall 323 in the vacuum thermal insulation layer for active thermal insulation. Before the sampling apparatus is lowered into the water, temperatures of the plurality of target water sampling layers are determined and correspondingly set as the target temperatures of the cooling heat-exchange modules 325 of the plurality of sampling bottles 32. The cooling heat-exchange modules 325 in different sampling bottles 32 automatically control the temperature based on the target temperature. The combination of active cooling and vacuum thermal insulation achieves efficient thermal insulation of seawater. The cooling end of the semiconductor cooling chip 3251 is closely attached to the outer wall surface of the inner bottle wall 323. The heat dissipation end of the semiconductor cooling chip 3251 conducts heat through a semiconductor heat exchange chip 3252, and the semiconductor heat exchange chip 3252 transfers heat with external circulating seawater by using a semiconductor heat-exchange water tank 3253. The seawater circulation heat exchange unit 6 enables a single circulation injection pump 61 to provide circulating seawater for the cooling heat-exchange modules 325 of multiple sampling bottles 32. The cooling heat-exchange modules 325 of different sampling bottles are connected in parallel via the second multi-channel distribution valve 62 and the third multi-channel distribution valve 63.

When the sampling apparatus is lowered into the water, the circulation injection pump 61 is activated. During the circulating water flow process, seawater first enters the circulation injection pump 61 via the first water inlet of the circulation injection pump 61 and then flows out via the second water outlet of the circulation injection pump 61. The outflowing seawater forms multiple channeled seawater flows in the second multi-channel distribution valve 62 and then enters the cooling heat-exchange modules 325 respectively via the seawater inlet pipes 64 which are connected to the seawater circulation inlets 327 of different sampling bottles 32. For multiple cooling heat-exchange modules 325 on a single sampling bottle 32, the circulating seawater is reused for heat exchange through series connection via the circulation pipeline 326. The seawater after heat exchange flows out via the seawater circulation outlet 328 of the sampling bottle 32. The seawater flowing out from different sampling bottles 32 is collected in the third multi-channel distribution valve 63 via the seawater outlet pipes 65, flows into the circulation injection pump 61 via the second water inlet, and is finally discharged via the first water outlet. The seawater circulation heat exchange unit 6 and cooling heat-exchange modules 325 are activated immediately after the apparatus is lowered into the water, enabling different sampling bottles 32 to quickly reach and stabilize at the temperature of the target seawater layer.

The sampling apparatus is lowered to the target water sampling layer, the sampling injection pump 41 and the automatic shutoff valve 42 are controlled to be activated, and a parameter of the flow rate controller 43 is set to control the injected seawater volume, allowing seawater to be slowly and isobarically injected into the liquid phase chamber of the sampling bottle 32. Since the pressure of the gas phase chamber of the sampling bottle 32 is equal to the external hydrostatic pressure, and the back pressure of the back pressure valve 34 is also equal to the pressure of the gas phase chamber, the sampling injection pump 41 only needs to provide minimal additional injection force. When the seawater volume reaches the preset target value, the automatic shutoff valve 42 closes automatically to prevent further seawater injection. After the automatic shutoff valve 42 closes, the sampling injection pump 41 stops working due to current self-protection caused by the blocked inlet, and the flow rate controller 43 also stops with the closure of the automatic shutoff valve 42. The rotation unit 2 rotates away from the position of the current sampling valve 31, and the current sampling valve 31 closes. This process is repeated to sample multiple target water sampling layers.

This embodiment addresses the issues of brief escape of dissolved gases in water and phased distortion of microorganisms that may result from passive sampling based on high pressure differences. Since the pressure of the gas phase chamber of the sampling bottle 32 is equal to the external hydrostatic pressure, and the back pressure of the back pressure valve 34 is also equal to the pressure of the gas phase chamber, the sampling injection pump 41 only needs to provide minimal additional injection force. The combination of the back pressure valve 34 and the second check valve 37 ensures that the high-pressure gas in the gas phase chamber of the sampling bottle 32 does not escape, external high-pressure seawater does not backflow, and high-pressure gas is slowly discharged. With the combination of the back pressure valve 34 and the check valve, the high-pressure gas in the gas phase chamber of the sampling bottle 32 does not escape, external high-pressure seawater does not backflow, and high-pressure gas is slowly discharged at specified times. Additionally, a single circulation injection pump 61, combined with deep low-temperature seawater, provides efficient heat exchange for the cooling heat-exchange modules 325 of multiple sampling modules 3, eliminating the need for additional cooling apparatuses and significantly reducing apparatus costs. The sampling bottle, with its double-layer wall design combined with cooling heat-exchange modules 325, integrates semiconductor cooling and vacuum thermal insulation to achieve stable and precise control of different seawater temperatures at various depths.

### Embodiment 3

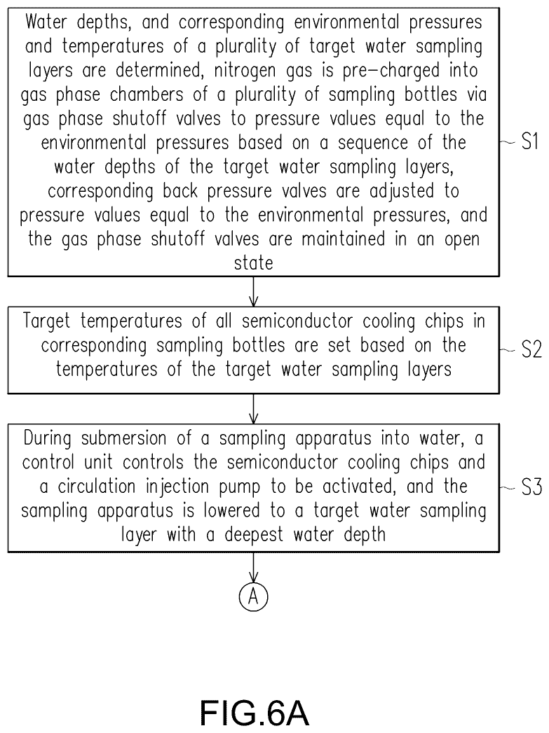

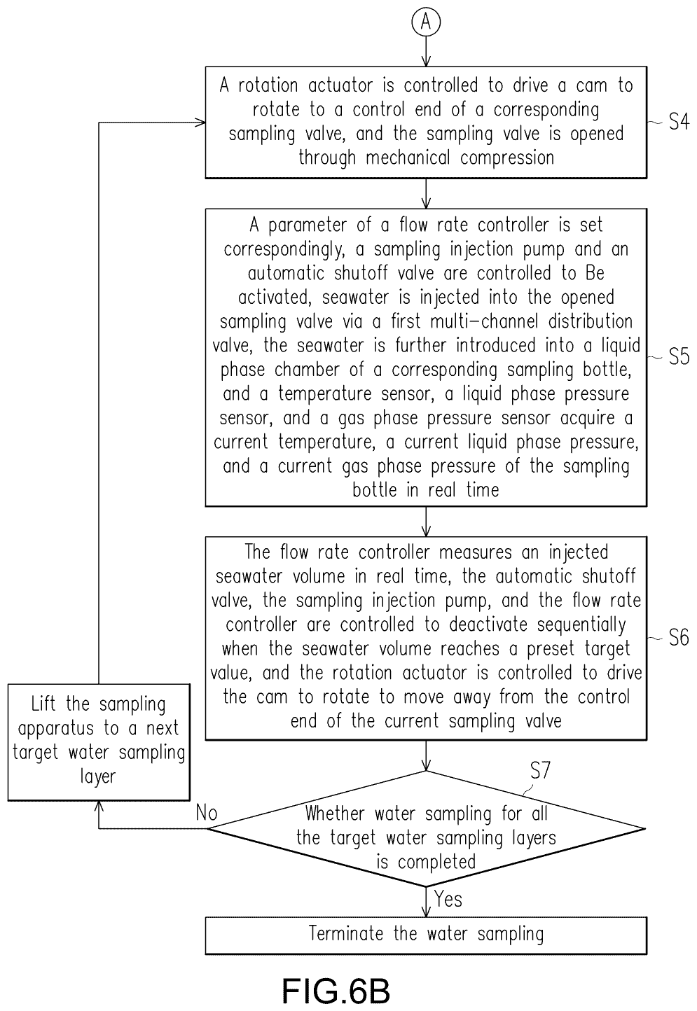

This embodiment of this application provides a multi-sequence seawater sampling method with thermal insulation and pressure retention, applied to the sampling apparatus described in Embodiment 1 or 2, as shown in FIG. 6A and FIG. 6B, and including:

S1: determining water depths, and corresponding environmental pressures and temperatures of a plurality of target water sampling layers, pre-charging nitrogen gas into the gas phase chambers of the plurality of sampling bottles correspondingly via the gas phase shutoff valves to pressure values equal to the environmental pressures based on a sequence of the water depths of the target water sampling layers, adjusting corresponding back pressure valves to pressure values equal to the environmental pressures, and maintaining the gas phase shutoff valves in an open state;
S2: setting target temperatures of all the semiconductor cooling chips in corresponding sampling bottles based on the temperatures of the target water sampling layers;
S3: during submersion of the sampling apparatus into water, controlling, by the control unit, the semiconductor cooling chips and the circulation injection pump to be activated, and lowering the sampling apparatus to a target water sampling layer with a deepest water depth;
S4: controlling the rotation actuator to drive the cam to rotate to the control end of a corresponding sampling valve, and opening the sampling valve through mechanical compression;
S5: setting a parameter of the flow rate controller correspondingly, controlling the sampling injection pump and the automatic shutoff valve to be activated, injecting seawater into the opened sampling valve via the first multi-channel distribution valve, further introducing the seawater into the liquid phase chamber of a corresponding sampling bottle, and acquiring, by the temperature sensor, the liquid phase pressure sensor, and the gas phase pressure sensor, a current temperature, a current liquid phase pressure, and a current gas phase pressure of the sampling bottle in real time;
S6: measuring, by the flow rate controller, an injected seawater volume in real time, controlling the automatic shutoff valve, the sampling injection pump, and the flow rate controller to be deactivated sequentially when the seawater volume reaches a preset target value, and controlling the rotation actuator to drive the cam to rotate to move away from the control end of the current sampling valve; and
S7: determining whether water sampling for all the target water sampling layers is completed; and if the water sampling for all the target water sampling layers is not completed, lifting the sampling apparatus to a next target water sampling layer and repeating steps S4 to S6; otherwise, terminating the water sampling.

After the lowering the sampling apparatus to a target water sampling layer with a deepest water depth, the method further includes:
introducing the seawater via the first water inlet of the circulation injection pump and discharging the seawater via the second water outlet; distributing the discharged seawater into a plurality of channeled seawater flows via the second multi-channel distribution valve, and introducing the plurality of channeled seawater flows into the cooling heat-exchange modules of different sampling bottles through the seawater inlet pipes from the seawater circulation inlets; and
collecting the seawater, after heat exchange through all the cooling heat-exchange modules of the sampling bottles, from the seawater circulation outlets into the third multi-channel distribution valve via the seawater outlet pipes; and introducing the collected seawater into the circulation injection pump via the second water inlet and discharging the seawater via the first water outlet. Identical or similar reference numerals correspond to identical or similar components.

Terms describing positional relationships in the drawings are for illustrative purposes only and should not be construed as limiting this patent.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

What is claimed is:

1. A multi-sequence seawater sampling method with thermal insulation and pressure retention, applied to a multi-sequence seawater sampling apparatus with thermal insulation and pressure retention, wherein the multi-sequence seawater sampling apparatus comprises an outer frame, a rotation unit, a multi-sequence sampling unit, a flow velocity regulation unit, a control unit, and a seawater circulation heat exchange unit; wherein
the multi-sequence sampling unit is disposed in the outer frame and comprises a plurality of sampling modules, and each of the sampling modules comprises a sampling valve, a sampling bottle, a gas phase shutoff valve, a back pressure valve that are connected in sequence, each of the sampling modules further comprises a temperature sensor, a liquid phase pressure sensor, and a gas phase pressure sensor;
the plurality of sampling valves are circumferentially distributed at a top of the outer frame, and control ends of the sampling valves all face the rotation unit;
the sampling bottle comprises an upper end cap, an outer bottle wall, an inner bottle wall, a piston, a plurality of cooling heat-exchange modules, a seawater circulation inlet, a seawater circulation outlet, and a lower end cap, wherein the upper end cap is disposed at one end of the outer bottle wall, the lower end cap is disposed at the other end of the outer bottle wall, the inner bottle wall is concentrically disposed with the outer bottle wall, and a vacuum thermal insulation layer is formed between the outer bottle wall and the inner bottle wall; the piston is disposed in the inner bottle wall and divides a cavity between the inner bottle wall, the upper end cap, and the lower end cap into a liquid phase chamber and a gas phase chamber, the outer bottle wall is provided with the seawater circulation inlet and the seawater circulation outlet that are communicated with each other, the plurality of cooling heat-exchange modules are uniformly distributed on an outer wall surface of the inner bottle wall, and control ends of the plurality of cooling heat-exchange modules are all connected to an output end of the control unit; and each of the cooling heat-exchange modules comprises a semiconductor cooling chip, and a control end of the semiconductor cooling chip is connected to the output end of the control unit;
the rotation unit is disposed at a center of the top of the outer frame, and an end of the rotation unit abuts against the control end of the sampling valve; the rotation unit comprises a rotation actuator and a cam; and the rotation actuator is disposed at the center of the top of the outer frame, the cam is disposed on the rotation actuator, and an end of the cam abuts against the control end of the sampling valve;
the flow velocity regulation unit is disposed in the outer frame and comprises a sampling injection pump, an automatic shutoff valve, a flow rate controller, and a first multi-channel distribution valve that are connected in sequence, wherein each water outlet end of the first multi-channel distribution valve is connected to a water inlet end of the sampling valve of one sampling module correspondingly;
the seawater circulation heat exchange unit comprises a circulation injection pump, wherein both a first water inlet and a first water outlet of the circulation injection pump are suspended, a second water outlet of the circulation injection pump is connected to the seawater circulation inlet of one sampling bottle correspondingly, and a second water inlet of the circulation injection pump is connected to the seawater circulation outlet of one corresponding sampling bottle correspondingly; and
the control unit is disposed in the outer frame, and the output end of the control unit is connected to control ends of the rotation unit, the sampling bottle, the sampling injection pump, and the flow rate controller; and
the method comprises:
S1: determining water depths, and corresponding environmental pressures and temperatures of a plurality of target water sampling layers, pre-charging nitrogen gas into the gas phase chambers of the plurality of sampling bottles correspondingly via the gas phase shutoff valves to pressure values equal to the environmental pressures based on a sequence of the water depths of the target water sampling layers, adjusting corresponding back pressure valves to pressure values equal to the environmental pressures, and maintaining the gas phase shutoff valves in an open state;
S2: setting target temperatures of all the semiconductor cooling chips in corresponding sampling bottles based on the temperatures of the target water sampling layers;
S3: during submersion of the sampling apparatus into water, controlling, by the control unit, the semiconductor cooling chips and the circulation injection pump to be activated, and lowering the sampling apparatus to a target water sampling layer with a deepest water depth;
S4: controlling the rotation actuator to drive the cam to rotate to the control end of a corresponding sampling valve, and opening the sampling valve through mechanical compression;
S5: setting a parameter of the flow rate controller correspondingly, controlling the sampling injection pump and the automatic shutoff valve to be activated, injecting seawater into the opened sampling valve via the first multi-channel distribution valve, further introducing the seawater into the liquid phase chamber of a corresponding sampling bottle, and acquiring, by the temperature sensor, the liquid phase pressure sensor, and the gas phase pressure sensor, a current temperature, a current liquid phase pressure, and a current gas phase pressure of the sampling bottle in real time;
S6: measuring, by the flow rate controller, an injected seawater volume in real time, controlling the automatic shutoff valve, the sampling injection pump, and the flow rate controller to be deactivated sequentially when the seawater volume reaches a preset target value, and controlling the rotation actuator to drive the cam to rotate to an empty position between two of the sampling valves; and
S7: determining whether water sampling for all the target water sampling layers is completed; and if the water sampling for all the target water sampling layers is not completed, lifting the sampling apparatus to a next target water sampling layer and repeating steps S4 to S6; otherwise, terminating the water sampling.

2. The multi-sequence seawater sampling method with thermal insulation and pressure retention according to claim 1, wherein each of the sampling bottles further comprises a circulation pipeline;
the seawater circulation inlet, the plurality of cooling heat-exchange modules, and the seawater circulation outlet form series connection via the circulation pipeline; and
a water outlet end of the sampling valve is connected to the upper end cap of the sampling bottle, and the lower end cap of the sampling bottle is connected to the gas phase shutoff valve.

3. The multi-sequence seawater sampling method with thermal insulation and pressure retention according to claim 2, wherein each of the cooling heat-exchange modules further comprises a semiconductor heat exchange chip and a semiconductor heat-exchange water tank;
a cooling end of the semiconductor cooling chip is disposed on the outer wall surface of the inner bottle wall, a heat dissipation end of the semiconductor cooling chip is connected to one end of the semiconductor heat exchange chip, and the other end of the semiconductor heat exchange chip is connected to the semiconductor heat-exchange water tank;
the semiconductor heat-exchange water tank is provided with a first port and a second port; and
a series connection path is formed by arranging the circulation pipeline between the first port of the semiconductor heat-exchange water tank of one cooling heat-exchange module and the second port of the semiconductor heat-exchange water tank of another cooling heat-exchange module, the first port of the semiconductor heat-exchange water tank of a cooling heat-exchange module at one end of the series connection path is connected to the seawater circulation inlet via the circulation pipeline, and the second port of the semiconductor heat-exchange water tank of a cooling heat-exchange module at the other end of the series connection path is connected to the seawater circulation outlet via the circulation pipeline.

4. The multi-sequence seawater sampling method with thermal insulation and pressure retention according to claim 3, wherein the seawater circulation heat exchange unit further comprises a second multi-channel distribution valve, a third multi-channel distribution valve, a plurality of seawater inlet pipes, and a plurality of seawater outlet pipes;
the second water outlet of the circulation injection pump is connected to a water inlet end of the second multi-channel distribution valve, each water outlet end of the second multi-channel distribution valve is connected to one end of one seawater inlet pipe, and the other end of each of the seawater inlet pipes is connected to the seawater circulation inlet of one sampling bottle; and
the second water inlet of the circulation injection pump is connected to a water outlet end of the third multi-channel distribution valve, each water inlet end of the third multi-channel distribution valve is connected to one end of one seawater outlet pipe, and the other end of each of the seawater outlet pipes is connected to the seawater circulation outlet of one sampling bottle correspondingly.

5. The multi-sequence seawater sampling method with thermal insulation and pressure retention according to claim 4, wherein each of the sampling modules further comprises a liquid phase shutoff valve; and
a water outlet end of the sampling valve is connected to one end of the liquid phase shutoff valve, and the other end of the liquid phase shutoff valve is connected to the sampling bottle.

6. The multi-sequence seawater sampling method with thermal insulation and pressure retention according to claim 5, wherein each of the sampling modules further comprises a first check valve and a second check valve;
the water outlet end of the sampling valve is connected to one end of the first check valve, and the other end of the first check valve is connected to one end of the liquid phase shutoff valve; and
the other end of the back pressure valve is connected to one end of the second check valve, and the other end of the second check valve is suspended.

7. The multi-sequence seawater sampling method with thermal insulation and pressure retention according to claim 2, wherein the temperature sensor and the liquid phase pressure sensor are both disposed at the upper end cap, the gas phase pressure sensor is disposed at the lower end cap; and
data output ends of the temperature sensor, the liquid phase pressure sensor, and the gas phase pressure sensor are all connected to a data input end of the control unit.

8. The multi-sequence seawater sampling method with thermal insulation and pressure retention according to claim 6, after the lowering the sampling apparatus to the target water sampling layer with the deepest water depth, further comprising:
introducing the seawater via the first water inlet of the circulation injection pump and discharging the seawater via the second water outlet;
distributing the discharged seawater to form a plurality of channeled seawater flows via the second multi-channel distribution valve, and introducing the plurality of channeled seawater flows into the cooling heat-exchange modules of different sampling bottles through the seawater inlet pipes from the seawater circulation inlets;
collecting the seawater, after heat exchange through all the cooling heat-exchange modules of the sampling bottles, from the seawater circulation outlets into the third multi-channel distribution valve via the seawater outlet pipes; and
introducing the collected seawater into the circulation injection pump via the second water inlet and discharging the seawater via the first water outlet.
